(19) United States
(12) Patent Application Publication
(10) Pub. No.: US 2022/0409640 A1
(43) Pub. Date: Dec. 29, 2022

(54) METHODS USING PHOSPHATIDYLSERINE POWDER

(71) Applicant: Frutarom Ltd, Migdal Haemeq (IL)

(72) Inventors: Asher Widberg, Haifa (IL); Yael Herzog, Gesher Haziv (IL); Itay Shafat, Yokneam Ilit (IL)

(73) Assignee: Frutarom Ltd, Migdal Haemeq (IL)

(21) Appl. No.: 17/929,461

(22) Filed: Sep. 2, 2022

Related U.S. Application Data
(62) Division of application No. 16/066,227, filed on Jun. 26, 2018, now abandoned.
(60) Provisional application No. 62/281,439, filed on Jan. 21, 2016.

(51) Int. Cl. A61K 31/685; A61K 8/02; A61Q 19/00; A23L 33/12; A61Q 19/08; A61K 8/55; A61K 9/14; A23J 7/00

(52) U.S. Cl. CPC A61K 31/685; A61K 8/022; A61Q 19/00; A23L 33/12; A61Q 19/08; A61K 8/553; A61Q 19/004; A61K 9/14; A23J 7/00; A23V 2250/1852; A61K 9/143; A61K 2800/92; A23V 2200/318; A61K 2800/412; A23V 2200/316; A23V 2002/00

(57) ABSTRACT

The present invention provides Phosphatidylserine (PS) preparations with PS having a predetermined particle size for use in sports nutrition and/or promoting skin health in a subject. The methods of the present invention include administering a daily dose of the PS preparation to improve sports performance and/or promote skin health in a subject.

### FIELD OF THE INVENTION

The present invention refers to uses of Phosphatidylserine (PS) preparations with predetermined particle size.

### BACKGROUND OF THE INVENTION

Phosphatidylserine (PS), which is the main acid phospholipid in mammalian plasma membrane, has been shown to play a key role in the functioning of neuron membranes and to alter multiple neurochemical systems (See, Vance et al. Metabolism and functions of phosphatidylserine. Prog. Lipid Res. 2005; 44:207-234). Supplementation of PS to animal models has been shown to attenuate neuronal effects of aging, as well as restoring normal memory in a range of tasks. In human, administration of PS to subjects with age-associated memory impairment, as well as to mild cognitive impairment patients, resulted with a consistent improvement of performance in memory tests. Studies conducted on children and adolescents tested the effects of PS supplementation on various cognitive and behavior aspects. These studies attest to the ability of PS to improve memory, attention and more in children, as well as to the safety of PS in children (See, Hirayama et al. Effect of phosphatidylserine administration on symptoms of attention-deficit/hyperactivity disorder in children. AgroFOOD industry hi-tech. 2006; 17:32-36). Moreover, adding PS to the nutrition of those dealing with sports was also shown to be beneficial, improving recovery following exercise, reducing muscle soreness and improving peak performance as measured by endurance of cyclists (See, Starks et al., The effects of phosphatidylserine on endocrine response to moderate intensity exercise. J Int Soc Sports Nutr 5, 11 (2008)).

Determinants of optimal sports performance include genetic background, optimal training and optimal nutrition. Understanding the latter fact, many turn to supplementation of ergogenic aids, not relying on their regular daily diet. These supplements include proteins, plant products and various other nutrients. Over the years, accumulating data indicates that addition of PS to the list of nutritional aids may also have benefits to those training in sports, improving recovery following exercise, reducing muscle soreness and improving peak performance. Thus, including PS in sports nutrition products may be advantageous.

The present invention provides PS preparations with predetermined particle size that confer beneficial effects for certain applications which are superior to those of conventional PS.

### SUMMARY OF THE INVENTION

The present invention provides a phosphatidylserine powder composition comprising a phosphatidylserine for use in one or both of improving sport performance in a subject and promoting skin health in a subject; wherein at least 80% (w/w) of the phosphatidylserine powder composition has a particle size of 500 microns or less. Preferably the particle size of the phosphatidylserine powder composition is 400 microns or less, more preferably 350 microns or less, even more preferably 300 microns or less, even more preferably 250 microns or less, even more preferably 200 microns or less, and most preferably 150 microns or less. The percentage by weight of the phosphatidylserine powder that satisfies the aforementioned particle size is preferably at least 85% (w/w), more preferably at least 90% (w/w), even more preferably at least 95% (w/w), and most preferable at least 97% (w/w). Preferably less than 20% (w/w) of the powder has particle size above 500 microns, at times less than 15% (w/w) at times less than 10% (w/w), at times less than 8% (w/w), at times less than 5% (w/w), at times less than 3% (w/w) and at times less than 1% (w/w).

In certain other non-limiting embodiments of the present invention, the phosphatidylserine powder composition is prepared from a natural source, synthetic source, semisynthetic source, or a combination thereof. In certain other non-limiting embodiments of the present invention, the phosphatidylserine is prepared using a monophasic reaction, a bi-phasic reaction, or a combination thereof. In certain other non-limiting embodiments of the present invention, the phosphatidylserine is prepared using a mono-phasic reaction and purified using an organic solvent. In certain other non-limiting embodiments of the present invention, the organic solvent is an alcohol solvent. In certain other non-limiting embodiments of the present invention, the phosphatidylserine is prepared using a bi-phasic reaction. In certain other non-limiting embodiments of the present invention, when mixed in water without processing by high pressure homogenization, the powder composition disperses more homogeneously and demonstrates reduced sedimentation in comparison to conventional PS powder preparations, or with a PS preparation in which more than 20% (w/w) of the PS preparation has a particle size of above 500 microns. In certain other non-limiting embodiments of the present invention, the powder composition is mixed with a powder nutritional, nutraceutical, or pharmaceutical composition or functional or medical food to form a mixture, and when the mixture is mixed with water without processing by high pressure homogenization, the mixture disperses more homogeneously and demonstrates reduced sedimentation compared to a mixture of a powder nutritional, nutraceutical, or pharmaceutical composition and conventional PS powder preparations or a PS preparation in which more than 20% (w/w) of the PS preparation has a particle size of above 500 microns. In certain other non-limiting embodiments of the present invention, the powder composition when mixed without processing by high pressure homogenization with a liquid nutritional, nutraceutical, or pharmaceutical composition or functional or medical food more homogeneously disperses and demonstrates reduced sedimentation compared to conventional PS powder preparations, or a PS preparation in which more than 20% (w/w) of the PS preparation has a particle size of above 500.

The present invention also provides a nutritional, pharmaceutical, or nutraceutical composition or a functional or medical food comprising any one of the phosphatidylserine powder compositions disclosed above.

In certain other non-limiting embodiments of the present invention, the nutritional or nutraceutical composition is a biscuit, pastry, cake, bread, cereal, bar, snack, pill, tablet, pellets, dragées, capsule, soft gel, syrup, baby formula, adult formula, sports formula, sport powders, sports bars, sports drinks, ready to use drinks, protein formula, smoothies, sports gels, shakes, medical nutrition product, candy, gummy, or confectionary. In certain other non-limiting embodiments of the present invention, the nutritional, nutraceutical, or pharmaceutical composition or functional or medical food maintains a uniform appearance. In certain other non-limiting embodiments of the present invention, the nutritional, nutraceutical, or pharmaceutical composition or functional or medical food maintains elastic stability during storage. In certain other non-limiting embodiments of the present invention, the bulk density of the phosphatidylserine powder composition in the nutritional, nutraceutical, or pharmaceutical composition or functional or medical food is 0.2 to 0.7 g/ml. In certain other non-limiting embodiments of the present invention, the dosage unit of the phosphatidylserine powder composition in the nutritional, nutraceutical, or pharmaceutical composition or functional or medical food is 10 mg to 1000 mg. In certain other non-limiting embodiments of the present invention, the nutritional, nutraceutical, or pharmaceutical composition or functional or medical food is a liquid. In certain other non-limiting embodiments of the present invention, the nutritional, nutraceutical, or pharmaceutical composition or functional or medical food is a powder. In certain other non-limiting embodiments of the present invention, the nutritional, nutraceutical, or pharmaceutical composition or functional or medical food contains at least one protein and at least one carbohydrate. In certain other non-limiting embodiments of the present invention, the phosphatidylserine powder is at least 0.05% (w/w) of the nutritional, nutraceutical, or pharmaceutical composition or functional or medical food. In certain other non-limiting embodiments of the present invention, the nutritional, nutraceutical, or pharmaceutical composition or functional or medical food comprises a phosphatidylserine powder composition prepared from a natural source, synthetic source, semisynthetic source, or a combination thereof. In certain other non-limiting embodiments of the present invention, the nutritional, nutraceutical, or pharmaceutical composition or functional or medical food comprises a phosphatidylserine powder composition prepared using a monophasic reaction, a bi-phasic reaction, or a combination thereof. In certain other non-limiting embodiments of the present invention, the nutritional, nutraceutical, or pharmaceutical composition or functional or medical food comprises a phosphatidylserine powder composition prepared using a mono-phasic reaction and purified using an organic solvent. In certain other non-limiting embodiments of the present invention, the organic solvent is an alcohol solvent. In certain other non-limiting embodiments of the present invention, the nutritional, nutraceutical, or pharmaceutical composition or functional or medical food further comprises another phospholipid.

The present invention also provides any one of the above mentioned phosphatidylserine power compositions for use in a method for one or both of improving sport performance in a subject and promoting skin health in a subject.

The present invention also provides any one of the above mentioned nutritional, pharmaceutical, or nutraceutical composition or a functional or medical food for use in a method for one or both of improving sport performance in a subject and promoting skin health in a subject.

The present invention also provides a method of using any one of the above mentioned phosphatidylserine powder compositions, the method comprising administering to a subject by oral ingestion a daily dose of the phosphatidylserine powder composition. In non-limiting embodiments of the present invention, the daily dose provides 100 mg to 1000 mg of phosphatidylserine. In non-limiting embodiments of the present invention, the daily dose provides 100 mg, 200 mg, 300 mg, 400 mg, 500 mg, 600 mg, 700 mg, 750 mg, 800 mg, 850 mg, 900 mg, 950 mg, or 1000 mg of phosphatidylserine. In non-limiting embodiments of the present invention, the daily dose is administered as a single daily dose. In non-limiting embodiments of the present invention, the daily dose is administered during two or three separate administrations per day. In non-limiting embodiments of the present invention, the individual is administered the daily dose for a period of at least 5 days, at times for 5 to 60 consecutive days at times for 14 days or more, at times for 21 days or more, at times for more than 50, 60, 100 or 180 days. In non-limiting embodiments of the present invention, the individual is administrated 100 mg, 200 mg, 300 mg, 400 mg, 500 mg, 600 mg, 700 mg, 750 mg, 800 mg, 850 mg, 900 mg, 950 mg, or 1000 mg of phosphatidylserine for 6 days a week, at times for 5 days a week, at times for 4, 3, 2 or only one day a week. In non-limiting embodiments of the present invention, at the end of the period sport performance is improved in the subject according to one or more parameters including: testosterone elevation, cortisol reduction, fatigue reduction, faster recovery from exercise, reduced muscle soreness, improved endurance, improved muscle strength, improved muscle size, enhanced athletic performance, improved sports-related decision making, improved selective attention of sports-related stimuli, improved concentration during sports events, and improved mental resilience during sports events. In non-limiting embodiments of the present invention, at the end of the period, skin health is promoted in the subject according to one or more parameters including: improved skin moisture, improved skin composure, decreased appearance of fine lines and wrinkles, reduced size of skin wrinkles, reduced photoaging and atopic skin and reduced photodamage score.

In non-limiting embodiments of the present invention, the individual is administered the daily dose for 5, 7, 10, 14, 15, 20, 21, 25, 28, 30, 35, 40, 45, 56 or 60, at time more than 14 days, at times more than 21 days, at times more than 50, 60 100 or 180 days of consecutive days. In non-limiting embodiments of the present invention, the invention provides a method of improving sport performance in the subject using any one of the above mentioned methods. In non-limiting embodiments of the present invention, the invention provides a method of promoting skin health in the subject. In non-limiting embodiments of the present invention, skin health is promoted in at least the subject's face.

### DETAILED DESCRIPTION OF THE INVENTION

The present invention provides PS powder composition comprising a PS in which the majority of the particles have a predetermined particle size for use in one or both of improving sport performance in a subject and promoting skin health in a subject.

The term “improving sport performance” or any lingual variations thereof as used herein should be understood to encompass improvement of at least one sport parameters. Non-limiting examples of such improvements are: testosterone elevation, cortisol reduction, fatigue reduction, faster recovery from exercise, reduced muscle soreness, improved endurance, improved muscle strength, improved muscle size, enhanced athletic performance, improved sports-related decision making, improved selective attention of sports-related stimuli, improved concentration during sports events, and improved mental resilience during sports events.

The term “fatigue reduction” or any lingual variations thereof as used herein should be understood to encompass one or more of general fatigue reduction, general physical fatigue, general mental fatigue, heart fatigue reduction, muscle fatigue reduction, aerobic fatigue, anaerobic fatigue, tiredness, exhaustion, weariness, felling worn-out, asthenia, lassitude, prostration, exercise intolerance, lack of energy and weakness. The term “faster recovery” or any lingual variations thereof as used herein should be understood to encompass one or more of general recovery following exercise, muscle recovery, mental recovery, restoration of energy, reduction of physical and mental stress, metabolic recovery, immediate recovery, short-term recovery and training recovery.

The term “enhanced athletic performance” or any lingual variations thereof as used herein should be understood to encompass one or more of enhanced running endurance, enhanced running speed, enhanced swimming endurance, enhanced swimming speed, enhanced jumping length, enhanced jumping height, enhanced cycling endurance, enhanced cycling speed, enhanced throwing distance, enhanced weight lifting, enhanced rowing endurance, enhanced rowing speed, enhanced walking endurance and enhanced walking speed.

The term “skin health” or any lingual variations thereof as used herein should be understood to encompass improvement of at least one skin parameters including skin moisture, skin composure, decreasing the appearance of fine lines and wrinkles, reducing the size of skin wrinkles, reducing photodamage score, protecting the skin from UV damage, photoaging and atopic skin.

In a further one of its aspects, the present invention provides PS powder composition comprising a PS in which the majority of the particles have a predetermined particle size for use in a method for one or both of improving sport performance in a subject and promoting skin health in a subject.

In a further one of its aspects, the present invention provides PS powder composition comprising a PS in which the majority of the particles have a predetermined particle size for one or both of improving sport performance in a subject and promoting skin health in a subject.

In a further one of its aspects, the present invention provides PS powder composition comprising a PS in which the majority of the particles have a predetermined particle size for use in the manufacture of a pharmaceutical, nutritional or a nutraceutical composition or a functional or medical food, for one or both of improving sport performance in a subject and promoting skin health in a subject.

In a further one of its aspects, the present invention provides a method comprising administering to a subject a PS powder composition comprising a PS in which the majority of the particles have a predetermined particle size, the method being for one or both of improving sport performance in a subject and promoting skin health in a subject.

According to another one of its aspects the invention provides PS powder composition comprising a PS in which the majority of the particles have a predetermined particle size for improving sport performance in one or more of an athlete, a professional athlete, a recreational athlete, a player in professional sports team, a player in recreational sports team, a player in professional sports, a player in recreational sports, a weight lifter, a cyclist, a swimmer, a rower and a walker. According to another one of its aspects the invention provides PS powder composition comprising a PS in which the majority of the particles have a predetermined particle size for improving skin health in one or more of: a person having dry skin, a person having wrinkled skin, a person exposed to UV light, a person with photo-damage, a person with abnormal skin composure, a person with atopic skin.

According to some embodiments the predetermined particle size is 500 microns or less, preferably 450 microns or less, preferably 400 microns or less, more preferably 350 microns or less or 300 microns or less, even more preferably 250 microns or less or 200 microns or less, and most preferably 150 microns or less.

According to some embodiments at least 80% (w/w) of the particles in the PS powder preparation have the predetermined size, at times at least 85% (w/w), at times at least 90% (w/w), at times at least 95% (w/w), at times at least 97% (w/w) and at times at least 99% (w/w).

The present invention provides phosphatidylserine powder composition comprising a phosphatidylserine, wherein at least 80% (w/w), at times at least 85% (w/w), at times at least 90% (w/w), at times at least 95% (w/w), at times at least 97% (w/w), and at times at least 99% (w/w) of the phosphatidylserine powder composition has a particle size of 500 microns or less.

The present invention provides phosphatidylserine powder composition comprising a phosphatidylserine, wherein less than 20% (w/w), at times less than 15% (w/w), at times less than 10% (w/w), at times less than 8% (w/w), at times less than 5% (w/w), at times less than 3% (w/w) and at times less than 1% (w/w) of the phosphatidylserine powder preparation has particle size of above 500 microns.

According to some embodiments at least 80% (w/w), at times at least 85% (w/w), at times at least 90% (w/w), at times at least 95% (w/w), at times at least 97% (w/w), and at times at least 99% (w/w) of the phosphatidylserine powder composition has a particle size of 450 microns or less.

According to some embodiments at least 80% (w/w), at times at least 85% (w/w), at times at least 90% (w/w), at times at least 95% (w/w), at times at least 97% (w/w), and at times at least 99% (w/w) of the phosphatidylserine powder composition has a particle size of 400 microns or less.

According to some embodiments at least 80% (w/w), at times at least 85% (w/w), at times at least 90% (w/w), at times at least 95% (w/w), at times at least 97% (w/w), and at times at least 99% (w/w) of the phosphatidylserine powder composition has a particle size of 350 microns or less.

According to some embodiments at least 80% (w/w), at times at least 85% (w/w), at times at least 90% (w/w), at times at least 95% (w/w), at times at least 97% (w/w), and at times at least 99% (w/w) of the phosphatidylserine powder composition has a particle size of 300 microns or less.

According to some embodiments at least 80% (w/w), at times at least 85% (w/w), at times at least 90% (w/w), at times at least 95% (w/w), at times at least 97% (w/w) and at times at least 99% (w/w) of the phosphatidylserine powder composition has a particle size of 250 microns or less.

According to some embodiments at least 80% (w/w), at times at least 85% (w/w), at times at least 90% (w/w), at times at least 95% (w/w), at times at least 97% (w/w) and at times at least 99% (w/w) of the phosphatidylserine powder composition has a particle size of 200 microns or less.

According to some embodiments at least 80% (w/w), at times at least 85% (w/w), at times at least 90% (w/w), at times at least 95% (w/w), at times at least 97% (w/w) and at times at least 99% (w/w) of the phosphatidylserine powder composition has a particle size of 150 microns or less.

Particle size was tested based on Particle Size Distribution Estimation by Analytical Sieving USP 786 method I. Using this test method, particle size is represented by the minimum square aperture through which the particle can pass. For the purposes of the present invention, this method may be used to determine particle size irrespective of what percentage by weight of the sample has a particle size larger than 75 microns. Mechanical sieving was performed using 100 grams of a dry powder sample. The sample was then sieved using US Sieve number 60 or EU sieve number 250. This sieve selection is able to test for a particle size of 250 microns or less. One of ordinary skill in the art is able to select the proper sieve in order to test for a variety of particle sizes. The cumulative percentage by weight of sample passing through the sieve is the percentage by weight of the sample having a particle size (or less) of the sieve size opening for the selected sieve. Samples were agitated using manual tapping as the agitation method and agitated for five minutes. Agitation cycles were repeated until the weight of the test sieved did not change by more than 5% of the previous weight on that sieve. Particle size results were disregarded if particles were observed to be aggregated after agitation and testing was repeated.

As used herein the terms “phosphatidylserine” and “PS” used interchangeably, should be understood to encompass a lipid of the general formula:

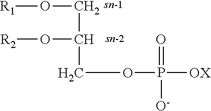

wherein the substituents, R₁ (substituent on position sn-1) and R₂ (substituent on position sn-2), are independent of each other and are selected from H or an acyl group selected from saturated, mono-unsaturated and polyunsaturated fatty acids and X is serine, i.e. —CH₂CH(COOH)NH₂.

The sn-1 and sn-2 positions as used herein and as indicated in above formula, refer to the respective carbon atoms on the glycerol backbone wherein R₁ and R₂, are substituted on the corresponding acyl groups.

In the present invention, the terms “substituted” and its lingual equivalents and the term “conjugated” and its lingual equivalents are interchangeably used and should be understood to encompass a fatty acid acyl covalently attached to the glycerophospholipid backbone of a serine glycerophospholipid of the invention. As noted above, the fatty acid may be attached to the sn-1 and/or sn-2 positions.

As used herein, the term “fatty acid” should be understood to encompass a carboxylic acid with a long unbranched aliphatic tail (chain), which is either saturated or unsaturated having one unsaturated bond (mono-unsaturated fatty acids) or two or more unsaturated bonds (poly-unsaturated fatty acids). When referring to a “fatty acid acyl” it should be understood to encompass an —C(═O)—R radical wherein R is a long unbranched aliphatic tail, which is either saturated or unsaturated having one unsaturated bond (mono-unsaturated fatty acids) or two or more unsaturated bonds (poly-unsaturated fatty acids).

Non-limiting examples of saturated fatty acids include: Butyric acid (Butanoic acid, C4:0), Caproic acid (Hexanoic acid, C6:0), Caprylic acid (Octanoic acid, C8:0), Capric acid (Decanoic acid, C10:0), Lauric acid (Dodecanoic acid, C12:0), Myristic acid (Tetradecanoic acid, C14:0), Palmitic acid (Hexadecanoic acid, C16:0), Stearic acid (Octadecanoic acid, C18:0), Arachidic acid (Eicosanoic acid, C20:0), Behenic acid (Docosanoic acid C22:0).

Non-limiting examples of unsaturated fatty acids include: Myristoleic acid (C14:1, ω-5), Palmitoleic acid (C16:1, ω-7), Oleic acid (C18:1, ω-9), Linoleic acid (C18:2, ω-6), Linolenic acid (C18:3) [Alpha-linolenic acid (C18:3, ω-3), Gamma-linolenic acid (C18:3, ω-6)], Eicosenoic acid (C20:1, ω-9), Arachidonic acid (C20:4, ω-6), Eicosapentaenoic acid (C20:5, ω-3), Erucic acid (C22:1, ω-9), Docosapentanoic acid (C22:5, ω-3) and Docosahexaenoic acid (C22:6, ω-3), Nervonic acid (C24:1, ω-9).

When referring to a “ . . . [fatty acid] conjugated to PS . . . ”, it should be understood to encompass a PS wherein a fatty acid acyl is conjugated at position sn-1 and/or position sn-2 of the phospholipid backbone (through the glycerol oxygen atom). In one embodiment a fatty acid is conjugated at position sn-1, and position sn-2 is either unsubstituted (e.g. having a hydrogen atom on the glycerol oxygen) or substituted with an acyl group selected from saturated, mono-unsaturated and polyunsaturated fatty acids, which may be the same or different from the substitution on position sn-1.

In another embodiment a fatty acid is conjugated at position sn-2 and position sn-1 is either unsubstituted (e.g. having a hydrogen atom on the glycerol oxygen) or substituted with an acyl group selected from saturated, mono-unsaturated and polyunsaturated fatty acids, which may be the same or different from the substitution on position sn-2.

The present invention also provides a nutritional, pharmaceutical, or nutraceutical composition or a functional or medical food comprising any one of the phosphatidylserine powder compositions disclosed above.

The subject invention envisages that PS powder preparations with particle size of 500 micron or less advantageously provide better stability and homogenous properties when incorporated into nutritional, nutraceutical, or pharmaceutical compositions or functional or medical food in comparison with PS preparations with particle size of more than 500 micron.

Furthermore, the invention provides PS preparations with particle size of 500 micron or less which are produced through a monophasic transphosphatidylation process, and advantageously possess even better stability and homogenous properties when incorporated into nutritional, nutraceutical, or pharmaceutical compositions or functional or medical food, in comparison with PS powder of the same particle size which is produced through a bi-phasic transphosphatidylation process.

The invention is advantageous compared to other existing commercial PS powders. Upon mixing with powder nutritional, nutraceutical, or pharmaceutical compositions or functional or medical food and dispersion in water, commercial PS powders/PS preparations in which more than 20% (w/w) of the powder has a particle size of above 500 microns tend to form non-homogenous mixtures with floating particle and sediments and reduced PS stability. Similar non-homogenous mixtures with reduced PS stability are also formed upon mixing commercial PS powders directly with water based nutritional, nutraceutical, or pharmaceutical compositions or functional or medical food. Furthermore, the addition of conventional PS powders or PS preparations in which more than 20% (w/w) of the powder has a particle size of above 500 microns to chewable matrixes, such as candies or gummies, tend to reduce the elasticity and stability of said chewable nutritional, nutraceutical, or pharmaceutical compositions or functional or medical food.

A nutritional composition as used herein can be any nutritional composition including, but not limited to: human milk fat substitute, infant formula, adult formula, dairy product, including milk and dairy drinks, milk powder, drinks, shakes, ice cream, biscuit, soy product, bakery, pastry, bread, cake, sauce, soup, prepared food, including prepared mashed vegetables and/or fruits, frozen food, condiment, confectionary, oil, fat, margarine, spread, filling, meat product, cereal, instant product, instant drink product, infant food, toddler food, bar, snack, candy, and chocolate product.

A nutraceutical composition as used herein can be any nutraceutical, which can be any substance that may be considered as a food or part of a food and provides medical or health benefits, including the prevention and treatment of diseases or disorders. Such nutraceutical compositions include, but are not limited to: a food additive, a food supplement, a dietary supplement, genetically engineered foods (such as for example vegetables, herbal products, and processed foods such as cereals, soups, and beverages), stimulant functional food, clinical nutrition product, medical food, and pharmafood. Dietary supplements may be delivered in the form of soft gel capsules, tablets, syrups, and other known dietary supplement delivery systems.

The pharmaceutical or nutraceutical compositions may be in any of the many dosage delivery forms commonly used in the art. Pharmaceutical or nutraceutical compositions suitable for oral administration may be presented as discrete dosage units (such as pills, tablets, pellets, dragées, capsules, or softgel), as a powder or granule, or as a solution, suspension, syrup, or elixir.

In a preferred embodiment the nutraceutical composition is a powder dietary supplement or functional food intended to be mixed with water, water based dietary supplement, water based functional food, or a chewable dietary supplement or functional food such as candy or gummy.

Preferably the nutritional or nutraceutical compositions further contain at least one protein and at least one carbohydrate components.

A functional food as used herein can be any functional food, including, but not limited to, dairy product, ice-cream, biscuit, soy product, bakery, pastry, cakes and bread, instant product, sauce, soup, prepared food, frozen food, condiment, confectionary, oils and fat, margarine, spread, filling, cereal, instant product, drinks and shake, infant food, bar, snack, candy and chocolate product.

Dietary supplements may be delivered in the form of pills, powders, tablets, pellets, dragées, capsules, soft gels, sachet, syrups, and other known dietary supplement delivery systems.

According to one embodiment the PS powder composition of the invention is added to medical foods.

A medical food as used herein is specially formulated and intended for the dietary management of a disease/disorder that has distinctive nutritional needs that cannot be met by normal diet alone.

According to one embodiment the present invention provide PS powder with bulk density with the range of 0.2-0.7 gr/ml, preferably 0.3-0.6 gr/ml, more preferably 0.35-0.55 gr/ml and most preferably 0.4-0.5 gr/ml.

In one embodiment, the PS powder preparation contains at least 10% w/w PS out of the preparation, preferably at least 20%, more preferably at least 30% or 40%, even more preferably at least 50% or 60% and most preferably at least 70% or 80%.

According to another embodiment the PS concentration out of the nutraceutical composition is between 0.05% to 10% w/w, preferably 0.1% to 7%, more preferably 0.5% to 5% and most preferably 1% to 4%.

According to a further embodiment the PS dose in a nutraceutical dosage unit is between 10 mg to 1000 mg, preferably between 30 mg to 500 mg, more preferably between 40 mg to 300 mg and most preferably between 50 mg to 100 mg of PS.

It should be noted that the PS preparation of the invention may also comprise other phospholipids, such as phosphatidylcholine (PC), phosphatidylethanolamine (PE), phosphatidyl-inositol (PI), phosphatidylglycerol (PG) and phosphatidic acid (PA), to which fatty acid acyls are covalently attached (bonded) at either or both of the sn-1 or sn-2 positions of the glycerol moiety of the phospholipid. The fatty acid conjugation profile of any of the above-noted polar lipids may be the same as, or different from, the fatty acid conjugation profile of PS, as disclosed herein.

The PS preparation of the invention is prepared from a natural, synthetic or semi-synthetic source or any combinations thereof. In an embodiment of the present invention, said natural source is derived from any one of plant (such as for example soy or sunflower), non-mammalian animal (such as for example krill, fish (such as for example Herring and blue Whiting)), or microorganism (such as for example bacteria) source or any combinations thereof.

Methods for preparing the PS powder preparation of the invention include, but are not limited to:
1. Bi-phasic transphosphatidylation reaction in which the reaction is carried out in a system containing aqueous and organic mediums. Lecithin is being dissolved in the organic phase while other ingredients such as phospholipase enzyme and serine are dissolved in water (as described in U.S. Pat. No. 5,700,668).
2. Monophasic transphosphatidylation reaction in which the reaction is carried out in an aqueous medium, the lecithin is dispersed during the reaction and other ingredients such as phospholipase enzyme and serine are dissolved in water (as described in U.S. Pat. No. 6,492,146 and WO05068644).

Preferably, following the transphosphatidylation reaction additional purification steps using a solvent are added to the procedure. Preferably the solvent is an alcohol solvent, more preferably an alcohol solvent with C1-C6 and most preferably an alcohol solvent with C2-C3. The solid is being dispersed with the solvent and stirred for one hour, preferably two hours and more preferably three hours and separation can be achieved using a variety of filtration techniques including but not limited to basket centrifuge, bowl centrifuge, decanter, etc., the solids are being washed more than once, preferably twice, more preferably three times and most preferably four times.

The obtained powder PS is dried using one of the following non-limiting technologies: vacuum oven, spray dryer, drum dryer, double cone dryer, paddle dryer, etc. and milled, optionally in one of the following non-limiting examples: Pin mill, Hammer mill, Cone mill, Jet mill, Boll mill, etc.

Preferably, following milling, the obtained PS powder is going through a sieving stage using one of the following non limiting technologies: air classifier, vibrating sieve, sifter, etc. to obtain PS powder preparation with predetermined particle size.

In one embodiment, powder PS is being sieved using 500 microns screen, preferably 400 microns screen, more preferably 350 microns or 300 microns, even more preferably 250 microns or 200 microns and most preferably 150 microns.

In another one of its aspects the invention provides a method of improving general health, cognitive functions and/or development, and/or improving and/or preventing a condition in a subject suffering from a cognitive disease or disorder comprising administering to a subject in need thereof the PS preparation of the invention.

The invention further provides a use of a preparation of the invention for the manufacture of a nutritional, nutraceutical or pharmaceutical composition or a medical or functional food for improving a condition in a subject suffering from a cognitive disease or disorder.

The term “cognitive disease or disorder” as used herein should be understood to encompass any cognitive disease or disorder. Non-limiting examples of such a cognitive disease or disorder are Attention Deficit Disorder (ADD), Attention Deficit Hyperactivity Disorder (ADHD), dyslexia, age-associated memory impairment, learning disorders, amnesia, mild cognitive impairment, cognitively impaired non-demented, pre-Alzheimer's disease, Alzheimer's disease, Parkinson's disease, pre-dementia syndrome, dementia, age related cognitive decline, cognitive deterioration, moderate mental impairment, mental deterioration as a result of aging, conditions that influence the intensity of brain waves and/or brain glucose utilization, stress, anxiety, depression, behavior disorders, concentration and attention impairment, mood deterioration, general cognitive and mental well-being, neurodegenerative disorders, hormonal disorders or any combinations thereof. In a specific embodiment, the cognitive disorder is memory impairment.

The term “improving a condition in a subject suffering from a cognitive disease or a cognitive disorder” as used herein should be understood to encompass: ameliorating undesired symptoms associated with a disease, disorder, or pathological condition; preventing manifestation of symptoms before they occur; slowing down progression of a disease or disorder; slowing down deterioration of a disease or disorder; slowing down irreversible damage caused in a progressive (or chronic) stage of a disease or disorder; delaying onset of a (progressive) disease or disorder; reducing severity of a disease or disorder; curing a disease or disorder; preventing a disease or disorder from occurring altogether (for example in an individual generally prone to the disease) or a combination of any of the above. For example, in a subject suffering from memory impairment, for example as a result of Alzheimer's Disease, symptoms including deterioration of spatial short-term memory, memory recall and/or memory recognition, focused and sustained attention, learning, executive functions and/or mental flexibility are improved by use of a lipid preparation of the invention.

According to one embodiment the PS preparation of the invention is dry blended with a powder nutraceutical prior to mixing the nutraceutical with water.

According to another embodiment, the powder PS of the invention is blended with water based nutraceutical.

In yet another aspect of the invention, the PS preparation is being added to chewable matrix, such as candy or gummy.

The numerical values provided herein are representative of those employed by the inventors for practicing and describing the preferred embodiments of the present invention. It should be appreciated that while these values are exemplary of preferred embodiments for practicing and describing the invention, those skilled in the art, in light of the present disclosure, will recognize that these values are not intended to necessarily require exact numerical precision and may be subject to a reasonable degree of variability without departing from the spirit and intended scope of the invention.

The following Examples are representative of techniques employed by the inventors in carrying out aspects of the present invention. It should be appreciated that while these techniques are exemplary of preferred embodiments for the practice of the invention, those of skill in the art, in light of the present disclosure, will recognize that numerous modifications can be made without departing from the spirit and intended scope of the invention.

### Example 1

A. Production of PS powder according to the invention (wherein at least 80% (w/w) of the PS powder composition has a particle size of 500 microns or less): 140 gr of an emulsion containing ethanol purified soy lecithin in water was mixed with a solution containing 41 gr L-serine, 35 ml acetate buffer and 0.55 gr phospholipase-D enzyme and stirred at 40° C. After 36 hours of stirring, the solution was filtered through a Buchner filter in order to remove all water soluble components. The solids were dispersed in 140 ml purified water for 30 minutes and filtered once again. 10 gr from the sample were dispersed in 40 ml ethanol and the solution was stirred for 30 minutes. The solution was filtered through a Buchner filter to separate between the solids and filtrate. The solids were collected from the filter and the same process was repeated two more times. Wet solids were dried in a vacuum dryer to obtain dry PS powder. Dry powder was milled together with 2% silicon dioxide to obtain free flowing powder and sieved through 250 microns screen in order to obtain powder in which 97% (w/w) of the powder had particle size of 250 microns or less.

B. Production of control PS powder (in which more than 20% (w/w) of the PS preparation has a particle size of above 500 microns): 140 gr of an emulsion containing ethanol purified soy lecithin in water was mixed with a solution containing 41 gr L-serine, 35 ml acetate buffer and 0.55 gr phospholipase-D enzyme and stirred at 40° C. After 36 hours of stirring, the solution was filtered through a Buchner filter in order to remove all water soluble components. The solids were dispersed in 140 ml purified water for 30 minutes and filtered once again. The wet solids were dried in a vacuum dryer to obtain dry PS powder with PS concentration of 58.8% measured using HPLC. Dry powder was milled together with 2% silicon dioxide to obtain free flowing powder in which more than 20% (w/w) of the powder had particle size above 500 microns.

### Example 2

Efficacy of the Preparation of the Invention for Improving Muscle Function, in Comparison with Conventional PS

Experimental design—Ten healthy human subjects are recruited for a double-blind, active-controlled trial. During the 8-week study period, the subjects receive either 600 mg conventional PS (Example 1B) or 600 mg of the preparation of the invention (Example 1A) per day.

During the 8 week study period the subjects undertake a series of weekly resistance training sessions. At baseline and endpoint of the study period the subjects undergo a series of tests aimed to measure the strength and size of their muscles, and fill a questioner aimed to record their muscle soreness following training and the speed at which they recover from training.

Following 8 weeks consumption of the preparation of the invention, the participants demonstrate increased muscle size and muscle strength, reduction in muscle soreness following training, improved recovery from training and leaner body mass in comparison with the group consuming conventional PS.

### Example 3

Efficacy of the Preparation of the Invention for Improving Parameters of Speed and Endurance, in Comparison with Conventional PS

Experimental design—Fifteen healthy male triathletes are recruited for a double-blind, active-controlled trial. During the 8-week treatment phase, the subjects receive either 600 mg of conventional PS (Example 1B) or 600 mg of the preparation of the invention (Example 1A) or 600 mg of a placebo per day.

During the 8-week treatment phase the subjects undertake a series of weekly training sessions which included running, swimming and/or cycling, the subjects undergo a series of tests aimed to measure the maximal speed of the various exercises and are measured for their VO2 80% max endurance.

Endurance may be measured by exercise induced time to exhaustion. Exercise induced time to exhaustion may be measured using an exercise test on an exercise apparatus, such as the electromagnetically braked cycle ergometer (Lode Excalibur Sport; Lode, Holland, or other commercially available equivalents thereof). First the work rates required for the staged intermittent exercise protocol are calculated for each subject by performing an incremental exercise test. Maximal oxygen uptake (VO2max) for each subject is identified using heart rate (Polar S810; Polar Electro Oy, Finland, or other commercially available equivalents thereof), and breath-by-breath respiratory parameters (Oxycon Pro; Jaeger, Germany, or other commercially available equivalents thereof) which are simultaneously recorded. Subjects are instructed to pedal at a constant cadence between 75 to 85 rpm. The work rate of the exercise is begun at 60 W and is incrementally increased by 30 W every 3 minutes until the subject is no longer able to achieve the cadence of 75 to 85 rpm. Using linear regression the work rates required for the staged intermittent exercise protocol are calculated for each subject. The staged intermittent exercise protocol requires three 10-minute stages of cycling at work rates that elicit 45%, 55% and 65% VO2max, respectively, followed by a final exercise stage at 85% VO2max that is continued until the subject is unable to maintain a cadence of 60 rpm despite verbal encouragement. Each exercise stage of the staged intermittent exercise protocol is interspaced with 5 minutes of passive rest periods. (See, Kingsley et al., Effects of Phosphatidylserine on Exercise Capacity during Cycling in Active Males. Med. Sci. Sports Exerc. 2006; 38(1): 64-71).

At the conclusion of the 8-week treatment phase, the participants that consume the preparation of the invention demonstrate an increase in maximal speed of one or more of the various exercises in comparison with both the conventional PS group and the placebo group. In addition, administration of the preparation of the invention leads to a greater increase in cycling endurance at VO2 80% max by the end of the treatment phase for the group that consume the preparation of the invention as compared to both the conventional PS group and the placebo group.

### Example 4

Efficacy of the Preparation of the Invention for Improving Skin Health and Composure, in Comparison with Conventional PS

Experimental design—Fifteen elderly human subjects with photodamaged skin are recruited for a double-blind, active-controlled trial. During the 12-week treatment phase, the subjects receive either 300 mg of conventional PS (Example 1B) or 300 mg of the preparation of the invention (Example 1A) or 300 mg of placebo per day.

Following 12 weeks consumption of the preparation of the invention, skin health is measured for one or more areas of the subject's skin including the face. Skin health is measured by examining one or more of the following parameters: skin moisture, skin composure, decreasing the appearance of fine lines and wrinkles, reducing the size of skin wrinkles, protecting the skin from UV damage, photoaging and atopic skin. Wrinkle size is measured according to the methods and procedures known in the art, for example the Profilometric method described in Hatzis et al., (Micron. 2004; 35(3):201-19). Photodamage is measured according to methods known in the art, for example the extensometry methods described in Marks & Edwards, (Br J Dermatol. 1992 September; 127 Suppl 41:7-13). In particular, skin moisture and composure is found to be improved, and photodamage score reduced. The level of improvement is found to an extent which is, surprisingly, greater than that in both the conventional PS group and placebo group.

1. A method for improving sport performance in a subject, for promoting skin health in a subject, or both, the method comprising administering to the subject by oral ingestion a composition comprising a phosphatidylserine powder wherein the phosphatidylserine powder comprises at least 20% by weight of a phosphatidylserine, and wherein at least 80% (w/w) of the phosphatidylserine powder has a particle size of 500 microns or less.

2. A method for improving sport performance in a subject, for promoting skin health in a subject, or both, the method comprising administering to the subject by oral ingestion a composition comprising a phosphatidylserine powder wherein the phosphatidylserine powder comprises at least 20% by weight of a phosphatidylserine, and wherein less than 20% (w/w) of the phosphatidylserine has a particle size of above 500 microns.

3. The method of claim 1, wherein the phosphatidylserine was produced using a monophasic reaction.

4. The method of claim 3, wherein the phosphatidylserine was purified using an organic solvent.

5. The method of claim 1, wherein the phosphatidylserine was produced using a bi-phasic reaction.

6. The method of claim 1, wherein the powder composition when mixed in water without processing by high pressure homogenization, disperses more homogeneously and demonstrates reduced sedimentation compared to a phosphatidylserine preparation in which more than 20% (w/w) of the phosphatidylserine preparation has a particle size of above 500 microns.

7. The method of claim 1, wherein the powder composition when mixed without processing by high pressure homogenization with a liquid nutritional, nutraceutical, or pharmaceutical composition or functional or medical food disperses more homogeneously and demonstrates reduced sedimentation compared to a phosphatidylserine preparation in which more than 20% (w/w) of the phosphatidylserine preparation has a particle size of above 500 microns.

8. The method of claim 1, wherein the phosphatidylserine powder composition is administered as a nutritional, nutraceutical, or pharmaceutical composition or functional or medical food.

9. The method of claim 8, wherein the phosphatidylserine powder is at least 0.05% (w/w) of the nutritional, nutraceutical, or pharmaceutical composition or functional or medical food.

10. The method of claim 2, wherein the phosphatidylserine was produced using a monophasic reaction.

11. The method of claim 10, wherein the phosphatidylserine was purified using an organic solvent.

12. The method of claim 2, wherein the phosphatidylserine was produced using a bi-phasic reaction.

13. The method of claim 2, wherein the powder composition when mixed in water without processing by high pressure homogenization, disperses more homogeneously and demonstrates reduced sedimentation compared to a phosphatidylserine preparation in which more than 20% (w/w) of the phosphatidylserine preparation has a particle size of above 500 microns.

14. The method of claim 2, wherein the powder composition when mixed without processing by high pressure homogenization with a liquid nutritional, nutraceutical, or pharmaceutical composition or functional or medical food disperses more homogeneously and demonstrates reduced sedimentation compared to a phosphatidylserine preparation in which more than 20% (w/w) of the phosphatidylserine preparation has a particle size of above 500 microns.

15. The method of claim 1, for improving sport performance in a subject, the method comprising administering to the subject by oral ingestion a daily dose of the phosphatidylserine powder for a period of at least 5 days, wherein the daily dose is 100 mg or more.

16. The method of claim 15, wherein at the end of the period sport performance is improved in the subject according to one or more parameters selected from the group consisting of: testosterone elevation, cortisol reduction, fatigue reduction, faster recovery from exercise, reduced muscle soreness, improved endurance, improved muscle strength, improved muscle size, enhanced athletic performance, improved sports-related decision making, improved selective attention of sports-related stimuli, improved concentration during sports events, and improved mental resilience during sports events.

17. The method of claim 1 for promoting skin health in a subject, the method comprising administering to the subject by oral ingestion a daily dose of the phosphatidylserine powder composition for a period of at least 5 days, wherein the daily dose is 100 mg or more.

18. The method of claim 17, wherein at the end of the period, skin health is promoted in the subject according to one or more parameters selected from the group consisting of: improved skin moisture, improved skin composure, decreased appearance of fine lines and wrinkles, reduced size of skin wrinkles, reduced photoaging and atopic skin and reduced photodamage score.

19. The method of claim 2 for improving sport performance in a subject, the method comprising administering to the subject by oral ingestion a daily dose of the phosphatidylserine powder composition for a period of at least 5 days, wherein the daily dose is 100 mg or more.

20. The method of claim 19, wherein at the end of the period sport performance is improved in the subject according to one or more parameters selected from the group consisting of: testosterone elevation, cortisol reduction, fatigue reduction, faster recovery from exercise, reduced muscle soreness, improved endurance, improved muscle strength, improved muscle size, enhanced athletic performance, improved sports-related decision making, improved selective attention of sports-related stimuli, improved concentration during sports events, and improved mental resilience during sports events.

21. The method of claim 2 for promoting skin health in a subject, the method comprising administering to the subject by oral ingestion a daily dose of the phosphatidylserine powder composition for a period of at least 5 days, wherein the daily dose is 100 mg or more.

22. The method of claim 21, wherein at the end of the period, skin health is promoted in the subject according to one or more parameters selected from the group consisting of: improved skin moisture, improved skin composure, decreased appearance of fine lines and wrinkles, reduced size of skin wrinkles, reduced photoaging and atopic skin and reduced photodamage score.
